(19) United States
(12) Patent Application Publication
(10) Pub. No.: US 2024/0225785 A1
(43) Pub. Date: Jul. 11, 2024

(54) DENTAL APPLIANCE WITH NON-AQUEOUS COMPOSITION

(71) Applicant: 3M INNOVATIVE PROPERTIES COMPANY, St. Paul, MN (US)

(72) Inventors: Yizhong Wang, Woodbury, MN (US); David K. Cinader, JR., Woodbury, MN (US); Joel D. Oxman, Minneapolis, MN (US)

(21) Appl. No.: 18/558,641

(22) PCT Filed: Apr. 25, 2022
(86) PCT No.: PCT/IB2022/053826

§ 371 Date: Nov. 2, 2023

Related U.S. Application Data
(60) Provisional application No. 63/201,900, filed on May 18, 2021.

(51) Int. Cl. A61C 7/08; A61C 19/06; A61K 6/20; A61K 8/04; A61K 8/19; A61K 8/21; A61K 8/24; A61K 8/25; A61K 8/92; A61Q 11/00

(52) U.S. Cl. CPC A61C 7/08; A61C 19/063; A61K 6/20; A61K 8/042; A61K 8/19; A61K 8/21; A61K 8/24; A61K 8/25; A61K 8/922; A61Q 11/00

(57) ABSTRACT

A system includes a dental appliance comprising a polymeric shell with a plurality of cavities for receiving one or more teeth. The system further includes a non-aqueous composition disposed in one or more of the cavities of the polymeric shell of the dental appliance. The non-aqueous composition includes an oil and a viscosity modifier. The non-aqueous composition has a viscosity of between 20 and 600 Pa*s at a shear rate 1/s.

### BACKGROUND

Compositions for use in dental appliances are described in U.S. Pat. App. Pub. 2020/0093576 and U.S. Pat. Nos. 10,682,300 and 10,772,821.

### BRIEF DESCRIPTION OF DRAWINGS

FIG. 1 is a schematic overhead perspective view of a system including a dental appliance and a non-aqueous solution.

FIG. 2 is a schematic overhead perspective view of a method in which for using a dental alignment tray is placed in the mouth of a patient to overlie teeth.

FIG. 3 is a schematic diagram of components of a kit including the dental appliance and non-aqueous solution.

FIG. 4 is a schematic illustration of a reclosable storage unit and dispenser configured to hold a dental appliance and dispense a non-aqueous solution into the tooth-retaining cavities in the dental appliance.

Like symbols in the drawings indicate like elements.

### DETAILED DESCRIPTION

Orthodontic treatments reposition misaligned teeth and improve bite configurations for improved cosmetic appearance and dental function. The teeth are repositioned by applying controlled forces to the teeth over an extended time period.

Teeth may be repositioned by placing a polymeric incremental position adjustment appliance, generally referred to as an orthodontic aligner or an orthodontic aligner tray, over the teeth of the patient for each treatment stage of an orthodontic treatment. The orthodontic alignment trays include a polymeric shell with a plurality of cavities for receiving one or more teeth. The individual cavities in the polymeric shell are shaped to exert force on one or more teeth to resiliently and incrementally reposition selected teeth or groups of teeth in the upper or lower jaw. A series of orthodontic aligner trays are provided for wear by a patient sequentially and alternatingly during each stage of the orthodontic treatment to gradually reposition teeth from one tooth arrangement to a successive tooth arrangement to achieve a desired tooth alignment condition. Once the desired alignment condition is achieved, an aligner tray, or a series of aligner trays, may be used periodically or continuously in the mouth of the patient to maintain tooth alignment.

In addition, orthodontic retainer trays may be used for an extended time period to maintain tooth alignment following the initial orthodontic treatment. Mouthguards and nightguards may also be used to temporarily protect teeth during athletic activities or to prevent damage caused by tooth-to-tooth contact or rubbing.

An orthodontic treatment or use of a retainer or a protective mouthguard may require that an orthodontic appliance remain in the mouth of the patient for up to 22 hours a day, over an extended time period of days, weeks, months, or even years.

Saliva is the mouth's primary defense against tooth decay. Healthy saliva flow helps prevent cavities by physically removing bacteria from the oral cavity before they can become attached to tooth and tissue surfaces and form a protected biofilm. The flow of saliva also helps dilute sugars and acids introduced by intake of food and beverages. The buffering capacity of saliva neutralizes acids and aids in the digestive process.

Placement of a dental appliance such as, for example, an orthodontic aligner tray; a retainer tray; a mouthguard, a nightguard, and the like, over the teeth of a patient can impede the natural flow of saliva around the teeth, which in some cases may increase the risk of tooth decay, particularly if the patient fails to consistently follow recommended regimens for tray cleaning and tooth brushing.

In addition, the nature of the action of orthodontic appliances dictates that the individual cavities in the polymeric shell be intentionally designed to fit imperfectly around select teeth of the patient, as this misfit causes the orthodontic aligner trays to deflect and exert correctional forces to those select teeth targeted for realignment. This misfit creates an air gap between the orthodontic appliance and some of the teeth of the patient. Even if the orthodontic appliance is made from a transparent polymeric material, air, liquids consumed by the patient, saliva, and entrained bubbles in the liquids, all residing in the air gap can cause the aligner tray to be more readily visible in the mouth of the patient, which creates a non-ideal aesthetic appearance. An orthodontic appliance that is substantially invisible over the teeth during treatment is most desirable for the patient.

Consequently, compositions, systems, and methods for mitigating the incidence of tooth decay

for wearers of such orthodontic appliances and/or improving the invisibility/aesthetic appearance of such orthodontic appliances are desirable.

As used herein, the singular forms “a”, “an”, and “the” include plural referents unless the content clearly dictates otherwise. As used in this specification and the appended embodiments, the term “or” is generally employed in its sense including “and/or” unless the content clearly dictates otherwise.

As used herein, the term “substantially”, unless otherwise specifically defined, means to a high degree of approximation (e.g., within +/−10% for quantifiable properties) but again without requiring absolute precision or a perfect match.

The term “or” is generally employed in its usual sense including “and/or” unless the content clearly dictates otherwise.

The term “and/or” means one or all of the listed elements or a combination of any two or more of the listed elements.

As used herein, the recitation of numerical ranges by endpoints includes all numbers subsumed within that range (e.g. 1 to 5 includes 1, 1.5, 2, 2.75, 3, 3.8, 4, and 5).

Unless otherwise indicated, all numbers expressing quantities or ingredients, measurement of properties and so forth used in the specification and embodiments are to be understood as being modified in all instances by the term “about.” Accordingly, unless indicated to the contrary, the numerical parameters set forth in the foregoing specification and attached listing of embodiments can vary depending upon the desired properties sought to be obtained by those skilled in the art utilizing the teachings of the present disclosure. At the very least, and not as an attempt to limit the application of the doctrine of equivalents to the scope of the claimed embodiments, each numerical parameter should at least be construed in light of the number of reported significant digits and by applying ordinary rounding techniques.

In general, the present disclosure is directed to a system including a dental appliance, such as an orthodontic aligner tray: an orthodontic retainer tray: a mouthguard, a nightguard, and the like, that includes a polymeric shell with tooth-retaining cavities. At least some of the tooth-retaining cavities contain a non-aqueous composition that includes an oil and a viscosity modifier. In some embodiments, the non-aqueous composition at least partially occupies the air gap between the dental appliance and the teeth, and has a chemical composition with a refractive index selected to render the orthodontic aligner tray less visible in the mouth of the patient.

The systems of the present disclosure offer a number of advantages. For example, filling the air gap with a composition that reduces the refractive index mismatch between the appliance and the air gap improves the aesthetic of the appliance when in the mouth. Additionally, as discussed in more detail below, the non-aqueous composition may exhibit a gel or gel-like consistency. Such consistency (combined with its non-aqueous property) renders the composition less likely to wash out and minimizes penetration of fluids consumed by the wearer into the tooth retaining cavities of the appliance.

Consequently, the aesthetics of the appliance can be maintained even when the wearer is consuming beverages (which is particularly helpful during social activities where beverages are served and it may be inconvenient to remove the appliance). Still further, when remineralizing additives are present, the non-aqueous compositions can mitigate the incidence of tooth decay for wearers of the orthodontic appliances. In some embodiments, a system 10 includes a dental appliance 100 as shown in FIG. 1, which

includes a thin polymeric shell 102 with tooth-retaining cavities 104 configured to fit over one or more of the teeth in the upper or lower jaw of a patient. In the embodiment shown in FIG. 1, the dental appliance 100 is an orthodontic aligner tray, but in other embodiments the dental appliance can be, for example, an orthodontic retainer tray, a mouthguard, or a nightguard. In the embodiment of FIG. 1, the tooth-retaining cavities 104 are shaped to receive and resiliently reposition one or more teeth from one tooth arrangement to a successive tooth arrangement. In other embodiments, a dental retainer tray may include tooth-retaining cavities 104 shaped to receive and maintain the position of the previously realigned one or more teeth, while a mouthguard or a nightguard includes tooth-retaining cavities 104 shaped to protect teeth during sports activities or to prevent teeth in the upper and lower jaws from rubbing against or striking one another and causing premature wear to a tooth surface.

The system 10 includes the dental appliance 100 with at least some of the tooth-retaining cavities 104 of the polymeric shell 102 including a non-aqueous composition 108. As used herein, the term “non-aqueous composition” refers to a composition having at least 50% by weight of an organic fluid, based on the total weight of the composition. In some embodiments, non-aqueous composition may include an organic fluid in an amount of at least 60 wt. %, at least 70 wt. %, at least 80 wt. %, at least 90 wt. %, or at least 95 wt. %, based on the total weight of the composition. In embodiments in which the non-aqueous composition includes both aqueous and non-aqueous fluids, the resulting composition may be homogeneous, i.e. a single-phase solution. As used herein, the term “organic fluid” refers to fluids comprising at least carbon and hydrogen, (e.g., hydrocarbons), but which may comprise other atoms such as oxygen, nitrogen, halogen, etc. Organic fluids may include, for example, one or more of petroleum, petroleum derivatives and petroleum distillates (e.g., mineral oil, lubricating oils, etc.), animal fats, plant oils, synthetic oils, hydroprocessed oils, and the like.

In some embodiments, the non-aqueous composition may be cosmetic. As used herein, the term cosmetic refers to compositions such as those described in the Federal Food, Drug and Cosmetic Act, sec. 201(i) (i.e., compositions not intended for use as a drug to cure or treat a disease in the mouth of the patient, but that includes components intended to be used in the polymeric shell 102 and in the mouth of a patient for cleansing, beautifying, or promoting attractiveness of the teeth, altering the appearance of the teeth, or reducing the visibility of the polymeric shell 102 when placed over the teeth). In some embodiments, the non-aqueous composition may be biocompatible. As used herein, the term biocompatible means that the non-aqueous composition includes components suitable for use in the mouth of a patient, and is not toxic, injurious, or physiologically reactive with the bodily fluids in the mouth or with the exposed surfaces of the teeth, and will not adversely react with commonly consumed foods and drinks. The disclosed non-aqueous compositions, one or more components in the compositions, or both, can be characterized as edible. Referring to a component or composition as edible means that the particular ingredient or composition is safe for daily, long-term ingestion at recommended use levels. In some embodiments, the GRAS (generally regarded as safe) list from the United States Food and Drug Administration (FDA) can be utilized to determine if a component is edible at the levels utilized in a composition.

In some embodiments, the non-aqueous composition may include an oil and a viscosity modifier.

In some embodiments, suitable oils include any food grade or edible oil such as food grade mineral oils, fish oils, or plant oils. As used herein, the term “plant oil” refers to any of various oils derived from plants or seeds and used in food products, medicinally, and industrially. Suitable plant oils may include, but are not limited to, cottonseed, flaxseed, soybeans, safflower, sunflower, sesame, canola, grapeseed, jatropha, jojoba, primula, poppy, camelina, cabbage, olive, coconut, palm, cotton, corn, soy, peanut, nut, and combinations thereof.

In some embodiments, oils may be present in the non-aqueous composition in an amount of between 70 and 90 wt. %, between 65 and 95 wt. %, or between 60 and 98 wt. %, based on the total weight of the non-aqueous composition.

In some embodiments, to provide a desired viscosity for a particular application, the non-aqueous composition may include a viscosity modifier. As used herein, the term “viscosity modifier” refers to any material that is compatible with the oil and can adjust the viscosity. Suitable viscosity modifiers for use in the non-aqueous compositions of the present disclosure may include oil compatible polymers (e.g., soluble or dispersible in the oil), silica compounds (including surface treated silica compounds), organic modified clay, or combinations thereof.

Oil compatible polymers dissolve, disperse, or swell in oil and thus modify the physical properties of the non-aqueous composition to provide properties such as, for example, gelation or thickening. Suitable biocompatible, oil compatible polymers include, cellulose derivative (e.g., methylcellulose, propyl cellulose, ethyl cellulose) acrylate polymers and copolymers (e.g., ethyl methacrylate polymer and copolymer, butyl acrylate polymer and copolymer), soilbean oil based polymer, and oliver oil based copolymer. In various embodiments, oil compatible polymers may be present in the non-aqueous composition in an amount of between 0.5 and 20 wt. %, between 1 and 15 wt. %, or between 5 and 10 wt. %, based on the total weight of the composition.

In some embodiments, the viscosity modifier may additionally, or alternatively, include a silica compound. Suitable silica compounds include, but are not limited to, fumed or precipitated silicas such as those available under the trade designation AEROSIL from Evonik Industries, Parsippany, NJ, and CAB-O-SIL from Cabot Corp, Alpharetta, GA. In some embodiments, the non-aqueous composition may include silica compounds in an amount of between 0.1 and 20 wt. %, between 5 and 18 wt. %, or between 8 and 15 wt. %, based on the total weight of the non-aqueous composition.

In some embodiments, viscosity modifiers may be present in an amount sufficient for the non-aqueous composition to form a gel, which in the present application refers to a flexible, viscous, liquid colloidal material that tends to cling to the dental appliance 100 such that a substantial portion of the gel remains in the tooth retaining cavities 104 until displaced by the teeth of the patient. In some embodiments, the gel is capable of swelling on contact with body fluids in the mouth, (or in fluids similar to body fluids such as physiological saline), but does not dissolve in water. The gels are substantially continuous, i.e., lacking a cellular or void structure (although minor defects such as entrapped air bubbles or fractures may be present), and thus generally are in a solid or semi-solid form. The term gel is used regardless of the state of hydration.

In some embodiments, the non-aqueous composition may have a viscosity selected to prevent rapid drainage from the tooth-retaining cavities 104 of the polymeric shell 102 when the dental appliance 100 being inserted into the mouth of the patient and the tooth retaining cavities 104 are positioned to overlie the teeth of the patient. For example, suitable drainage-resistant nonaqueous compositions have been found to have a viscosity of 1 to 1500 Pa*s, 10 to 1000 Pa*s, or 20 to 600 Pa*s at a shear rate of 1/s; 0.1 to 200 Pa*s, 0.5 to 100 Pa*s, or 2 to 50 Pa*s, at shear rate of 10/s; or 0.01 to 20 Pa*s, 0.1 to 10 Pa*s, or 0.5 to 5 Pa*s at a shear rate of 100/s.

In some embodiments, the nonaqueous compositions exhibit shear thinning behavior. For purposes of the present application, shear thinning behavior means that as the shear rate increases, the viscosity of the composition decreases (i.e., shear rate and viscosity are inversely proportional).

As used herein, viscosity is determined in accordance with the shear viscosity measurements of the Examples section of the present application.

In various embodiments, viscosity modifiers (including oil compatible polymers, silica compounds, and other viscosity modifiers) may be present in the non-aqueous composition in an amount of between 0.5 and 20 wt. %, between 1 and 15 wt. %, or between 5 and 10 wt. %, based on the total weight of the composition.

In some embodiments, the aqueous liquid 108 in the system 10 may have a refractive index selected to minimize the refractive index difference between the polymeric shell of the dental appliance and the air gap between the teeth of a patient and the dental appliance occupied by the aqueous liquid. This matching of refractive index enhances the optical properties of the system 10 by reducing glare and reflectance at the surface of the teeth of the patient. Glare is defined herein as the average reflectance over a range of 450-650 nanometers and reflectance is defined herein as the process where a fraction of the radiant flux incident on a surface is returned into the same hemisphere whose base is the surface and which contains the incident radiation (see Handbook of Optics, 2^{nd} ed., McGraw-Hill, Inc., 1995). In some embodiments, the refractive index of the aqueous liquid 108 is selected to minimize both

the appearance of air bubbles entrained in the aqueous liquid, and the appearance of the dental appliance 100 overlying the teeth of the patient. The selection of the refractive index of the aqueous liquid 108 depends at least in part on the refractive index of the polymeric material of the polymeric shell 102. In some embodiments, the polymeric shell 102 may be a polymeric material having refractive indexes (RI) in the range of 1.48 to 1.65. For example, polymethyl(meth)acrylate (PMMA) has a RI of 1.489;

polycarbonate has a RI of 1.585; and polyethylene terephthalate (PET) has a RI of 1.64. In such embodiments, the non-aqueous composition may have a refractive index of between 1.10 and 1.65 or between, 1.20 and 1.55 and the refractive index of the polymeric material may differ from that of the nonaqueous composition by less than 30%, less than 20%, less than 15%, or less than 10%. In some embodiments, if the polymeric shell is PET having a RI of about 1.6, to minimize the appearance of the dental appliance over the teeth of the patient, the refractive index of the nonaqueous composition may be greater than about 1.3, or greater than about 1.33, or greater than about 1.34 (±0.01). The refractive index of the aqueous liquid 108 can be measured by, for example, a refractometer such as those available from Bausch & Lomb, Rochester, NY. As used herein, the refractive index of material shall be as taken at room temperature and when the material is free of visible bubbles.

In some embodiments, the non-aqueous composition of the present disclosure may be colorless (or at least substantially colorless) and transparent (or at least substantially transparent) to visible light with a wavelength of 400-750 nm, and as such, when free of visible bubbles, is not visually detectable against the teeth of the patient. In some embodiments, the polymeric shell 102 and the aqueous liquid 108 of the system 10 transmit at least 60%, or at least 80%, or at least 90%, of incident light with a wavelength of about 400-750 nm, which can render the system 10 substantially invisible when the polymeric shell 102 is positioned over the teeth of the patient.

In some embodiments, the non-aqueous composition can include one or more additives that provide a tooth re-mineralizing benefit for the patient (alone or in combination with one or more minerals that may be useful or beneficial for ingestion or oral health). In some embodiments, such additives may include suitable fluorides such as inorganic fluoride sources such as sodium fluoride, sodium monofluoride, stannous fluoride, calcium fluoride and the like, as well as organic fluoride sources such as tetraalkylammonium tetrafluoroborate salts and amine hydrofluoric acid salts, and mixtures and combinations thereof.

Illustrative optional minerals that can be included in the disclosed compositions can include calcium (Ca), phosphorus (P), magnesium (Mg), iron (Fe), strontium (Sr), zinc (Zn), potassium (K), or combinations thereof. In some embodiments, which are not intended to be limiting, some minerals can be provided by including magnesium chloride (MgCl₂), calcium chloride (CaCl₂), strontium chloride, zinc chloride, zinc gluconate, potassium nitrate, potassium phosphate dibasic (KH₂PO₄), or combinations thereof.

In some embodiments, the non-aqueous composition can be described by the pH thereof, the stability thereof, various other properties, or combinations thereof. In various embodiments, the non-aqueous composition may have a pH suitable for use in the mouth of a patient, and may be selected to aid in neutralizing oral acids from food and bacteria present in the mouth of the patient. In various embodiments, for example, the non-aqueous composition may have a pH of about 4.5 to about 9.5, or about 6.0 to about 8.0 (±0.1), or about 7.1 to about 7.35, or about 7.1. The composition can naturally have such a pH or can be buffered to have a pH in a useful, e.g., a “neutral” range.

In some embodiments, the non-aqueous composition can have desired stability properties. The stability of a composition can include microbiological stability, physical stability, or combinations thereof. In some embodiments, the non-aqueous composition may be microbiologically stable for at least 6 months, in some embodiments 1 year, in some embodiments greater than 2 years.

In some embodiments, the non-aqueous composition can also include additional components such as, for example, sweeteners, humectants, mineral salts, buffering components, flavorants, preservative agents, or combinations thereof. Other optional beneficial ingredients can also be included at appropriate levels such as, aloe vera (multi-benefit), folic acid (related to B12), hyaluronic acid (lubricating, healthy skin), ceramides (healthy skin), arginine, betaines or oxygenated glycerol triesters, vitamin E (antioxidant and preservative), vitamin B12 (healthy skin, etc.), EDTA, cetyl pyridinium chloride, chlorhexidine, other antiseptics, and combinations thereof.

In some embodiments, the shell 102 of the orthodontic appliance 100 may be an elastic polymeric material that generally conforms to a patient's teeth, and may be transparent, translucent, or opaque. In some embodiments, the shell 102 may be a clear or substantially transparent polymeric material that may include, for example, one or more of amorphous thermoplastic polymers, semi-crystalline thermoplastic polymers and transparent thermoplastic polymers. In some embodiments, the shell 102 includes a material chosen from polyurethane, polycarbonate, acrylic, polysulfone, polypropylene, polyester, copolyester, polypropylene/ethylene copolymer, cyclic olefin polymer/copolymer, poly-4-methyl-1-pentene or polyester/polycarbonate copolymer, styrenic polymeric materials, polyamide, polymethylpentene, polyetherketone and combinations thereof. In another embodiment, the shell 102 may be chosen from clear or substantially transparent semi-crystalline thermoplastic, crystalline thermoplastics and composites, such as polyamide, polyethylene terephthalate. polybutylene terephthalate, polyester/polycarbonate copolymer, polyolefin, cyclic olefin polymer, styrenic copolymer, polyetherimide, polyetherketone, polyethersulfone, polytrimethylene terephthalate, and mixtures and combinations thereof. In some embodiments, the shell 102 is a polymeric material chosen from polyethylene terephthalate, polyethylene terephthalate glycol (PETg), polycyclohexylenedimethylene terephthalate glycol, poly(meth)acrylates (which include polymethacrylates and polyacrylates), and mixtures and combinations thereof. One example of a commercially available material suitable as the elastic polymeric material for the shell 102, which is not intended to be limiting, is PETg. Suitable PETg resins can be obtained from various commercial suppliers such as, for example, Eastman Chemical, Kingsport, TN; SK Chemicals, Irvine, CA; DowDuPont, Midland, MI; Pacur, Oshkosh, WI; and Scheu Dental Tech, Iserlohn, Germany.

In some embodiments, the shell 102 may be made of a single polymeric material, or may include multiple layers of different polymeric materials.

In one embodiment, the shell 102 may be a substantially transparent polymeric material. In this context, the term substantially transparent refers to materials that pass light in the wavelength region sensitive to the human eye (about 400 nm to about 750 nm) while rejecting light in other regions of the electromagnetic spectrum. In some embodiments, the reflective edge of the polymeric material selected for the shell 102 should be above about 750 nm, just out of the sensitivity of the human eye.

The orthodontic appliance 100 may be made using a wide variety of techniques. A plurality of cavities 104 may be formed in a substantially flat sheet of polymeric material to form the orthodontic appliance 100, wherein the cavities are configured to receive one or more teeth. The cavities 104 may be formed by any suitable technique, including thermoforming, laser processing, chemical or physical etching, and combinations thereof.

In another embodiment, the shell-like orthodontic dental appliance 100 may be formed using a three-dimensional (3D) printing process (e.g. additive manufacturing), such as stereolithography.

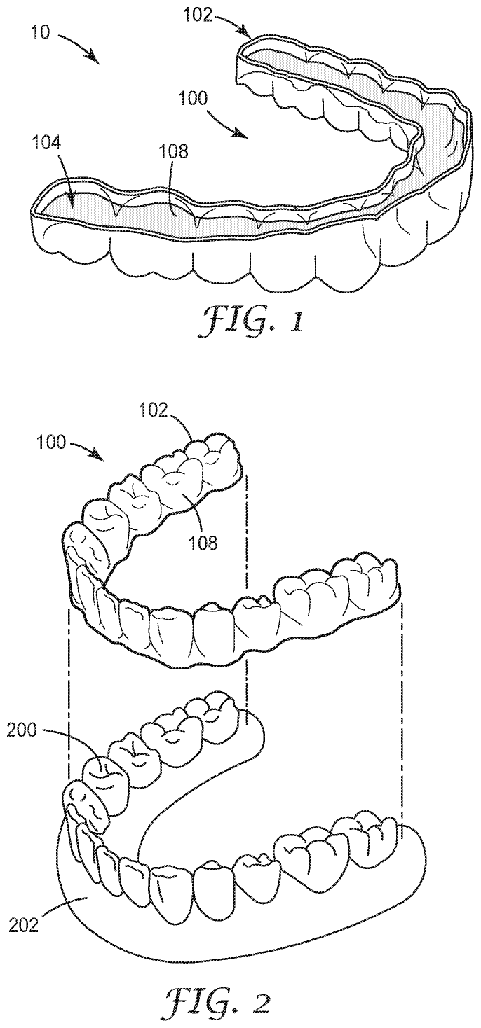

Referring now to FIG. 2, the shell 102 of the orthodontic appliance 100 may generally conform to a patient's teeth 200, but may be slightly out of alignment with the patient's initial maloccluded tooth configuration. In some embodiments, the shell 102 may be one of a group or a series of shells having substantially the same shape or mold, but which are formed from different materials to provide a different stiffness or resilience as needed to move the teeth of the patient. In this manner, in one embodiment, a patient or a user may alternately use one of the orthodontic appliances during each treatment stage depending upon the patient's preferred usage time or desired treatment time period for each treatment stage.

In some embodiments, the system 10 may include no wires or other means for holding the shell 102 over the teeth 200, but in some embodiments, it may be desirable or necessary to provide individual anchors on teeth with corresponding receptacles or apertures in the shell 102 so that the shell 102 can apply a retentive or other directional orthodontic force on the tooth which would not be possible in the absence of such an anchor.

In some embodiments, the shells 102 may be customized, for example, for day time use and night time use, during function or non-function (chewing vs. non-chewing), during social settings (where appearance may be more important) and nonsocial settings (where the aesthetic appearance may not be a significant factor), or based on the patient's desire to accelerate the teeth movement (by optionally using the more stiff appliance for a longer period of time as opposed to the less stiff appliance for each treatment stage). For example, in one aspect, the patient may be provided with a clear orthodontic appliance that may be primarily used to retain the position of the teeth, and an opaque orthodontic appliance that may be primarily used to move the teeth for each treatment stage. Accordingly, during the day time, in social settings, or otherwise in an environment where the patient is more acutely aware of the physical appearance, the patient may use the clear appliance. Moreover, during the evening or night time, in non-social settings, or otherwise when in an environment where physical appearance is less important, the patient may use the opaque appliance that is configured to apply a different amount of force or otherwise has a stiffer configuration to accelerate the teeth movement during each treatment stage. This approach may be repeated so that each of the pair of appliances are alternately used during each treatment stage.

Referring again to FIG. 2, the orthodontic appliance 100 and the non-aqueous composition 108 may form the dental alignment system. For example, a patient may be provided with a series of a plurality of incremental orthodontic appliances 100, each formed from the same or a different material, for each treatment stage of orthodontic treatment. The orthodontic appliances may be configured such that each successive member of the series incrementally repositions individual teeth 200 in an upper or lower jaw 202 of a patient from a maloccluded position to a desired non-occluded position. In some embodiments, the cavities 104 are configured such that selected teeth will be repositioned, while others of the teeth will be designated as a base or anchor region for holding the repositioning appliance in place as it applies the resilient repositioning force against the tooth or teeth intended to be repositioned.

In some embodiments, prior to placement of the orthodontic appliance 100 over the teeth, the non-aqueous composition 108 may be applied in some or all of the cavities 104 in the polymeric shell 102. The non-aqueous composition 108 may be applied in the cavities 104 in an amount sufficient such that when the orthodontic appliance 100 is placed over the teeth, the non-aqueous composition 108 occupies and remains in the air gap between the polymeric shell 102 and the teeth.

As noted above, in some embodiments the non-aqueous composition 108 may be in the form of a gel, and may have a viscosity selected such that all or a substantial amount of the gel remains in the cavities 104 as the polymeric shell 102 is placed over the teeth and the teeth are inserted into the cavities 104. As the teeth enter the cavities 104, a portion of the gel is displaced, but sufficient gel remains to substantially fill the air gap between the polymeric shell 102 and the teeth.

In some embodiments, the non-aqueous composition 108 may be dispensed from a collapsible tube-like container, a syringe, or an applicator and manually applied to the cavities 104 prior to insertion of the polymeric shell 102 over the teeth. In another embodiment, an automatic dispenser may be used to dispense a controlled amount of the non-aqueous composition 108 into the cavities 104.

Placement of the dental appliance 100 over the teeth 200 applies controlled forces in specific locations to gradually move the teeth into the new configuration. Repetition of this process with successive dental appliances having different configurations eventually moves a patient's teeth through a series of intermediate configurations to a final desired configuration.

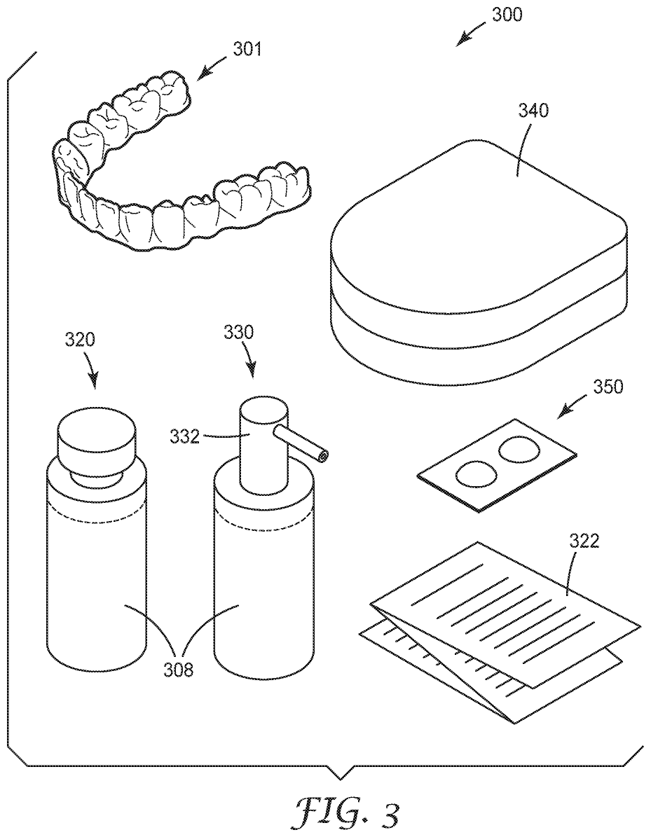

Referring to FIG. 3, in various embodiments, the system may be supplied in the form of a kit 300 including the dental appliance 301 and the non-aqueous composition 308. In some embodiments, the non-aqueous composition 308 may be supplied in a container 320 such as, for example, a squeezable bottle or a collapsible tube, along with instructions 322 for proper application to the dental appliance 301.

In another embodiment, the non-aqueous composition 308 may be supplied in a dispenser 330, wherein the dispenser 330 includes for example, a syringe, a trigger-operated gun, or a pump 332 configured to dispense a predetermined amount of the composition for each insertion of the dental appliance 301 into the mouth of the patient. In another embodiment, the kit may optionally include additional items such as, for example, a storage case 340, which may serve as a holder or an automated cleaning apparatus for temporary storage of the dental appliance 301 while not the dental appliance is not in the mouth of the patient, liquid cleaning or disinfecting solutions or solid tablets 350 dissolvable in water for use with the storage case or automated cleaning apparatus, a charger for the automated dispenser or cleaning apparatus, instructions for use, and the like.

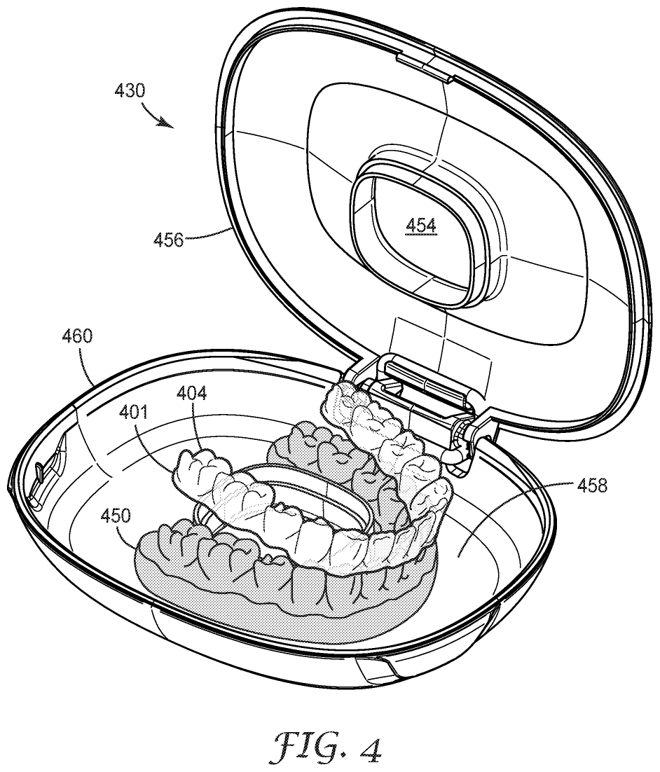

As shown in FIG. 4, in some embodiments, a re-closable storage unit and dispenser 430 includes a foam applicator pad 450 shaped to hold a shell-like dental appliance 401 including tooth-retaining cavities 404. The storage unit and dispenser 430 further includes an opening 454 in a hinged cover 456. When the non-aqueous composition (not shown in FIG. 4) is dispensed into, for example, a reservoir 458 in a bottom portion 460 of the dispenser 430, or directly on to the applicator pad 450, the non-aqueous composition collects in the reservoir 458. The foam applicator pad 450 absorbs the non-aqueous composition from the reservoir 458. When the cover 456 is closed and engages the bottom portion 460 of the dispenser 430, the dental appliance 401 is pressed against the foam applicator pad 450. The foam applicator pad 450 dispenses a predetermined measured amount of the non-aqueous composition into the cavities 404 of the dental appliance 401.

The systems and methods of the present disclosure will now be further described in the following non-limiting examples.

### EXAMPLES

TABLE 1
Materials

| Component | Supplier |
|---|---|
| Canola Oil (MAZOLA) | Distributed by ACH Food Companies, |
| | INC. Memphis, TN 38016, USA |
| AEROSIL R812S (fumed silica) | Evonik (Parsippany, NJ, USA). |
| AEROSIL 200 Pharma (fumed silica) | Evonik (Parsippany, NJ, USA). |
| Sodium Fluoride | Phibro Corporation, Neaneck, NJ |
| Tricalcium phosphate (beta-TCP) | Budenheim USA Inc., Mansfield, Ohio |
| Calcium carbonate USP; (CALESSENCE 300 | Specialty Minerals Inc, Adams, MA |
| Precipitated Calcium Carbonate (PCC)) | |

The following oil gel Examples were prepared by adding all ingredients in a mixing cup and speed mixing at a speed of 3000 rpm for two minutes and two cycles of mixing.

TABLE 2
Examples in Weight (grams)

| Component | Ex. 1 | Ex. 2 | Ex. 3 | Ex. 4 | Ex. 5 | Ex. 6 |
|---|---|---|---|---|---|---|
| Canola Oil | 45.00 | 46.00 | 45.00 | 45.00 | 45.00 | 45.00 |
| AEROSIL R812S | 7.00 | — | — | — | — | — |
| AEROSIL A200 | — | 4.00 | 5.00 | 5.00 | 5.00 | 5.00 |
| Sodium Fluoride | — | — | — | 0.50 | 0.50 | 0.50 |
| Tricalcium phosphate | — | — | — | — | 0.05 | 0.05 |
| Calcium carbonate | — | — | — | — | | 0.10 |
| Total Weight (Grams) | 52.00 | 50.00 | 50.00 | 50.50 | 50.55 | 50.65 |

TABLE 3
Examples in Weight Percent

| Component | Ex. 1 | Ex. 2 | Ex. 3 | Ex. 4 | Ex. 5 | Ex. 6 |
|---|---|---|---|---|---|---|
| Canola Oil | 86.54 | 92.00 | 90.00 | 89.11 | 89.02 | 88.85 |
| AEROSIL R812S | 13.46 | — | — | — | — | — |
| AEROSIL A200 | — | 8.00 | 10.00 | 9.90 | 9.89 | 9.87 |
| Sodium Fluoride | — | — | — | 0.99 | 0.99 | 0.99 |
| Tricalcium phosphate | — | — | — | — | 0.10 | 0.10 |
| Calcium carbonate | — | — | — | — | — | 0.20 |
| Total % | 100.00 | 100.00 | 100.00 | 100.00 | 100.00 | 100.00 |

The shear viscosity of the Example formulations was measured at room temperature on an AR-G2 Magnetic Bearing Rheometer from TA Instruments Ltd., with parallel plate fixture. Approximately 1.4 mL of example formulation was placed between the plates and the gap of the plates was set to 1 mm for the measurement at room temperature. The viscosity in units of Pascal-second was recorded at various shear rates for Examples Ex. 2-Ex. 6 and reported in Table 4. Two replicates for each formulation were conducted and the average was reported.

TABLE 4
Viscosity (Pa-s) of Examples at Various Shear Rates

| shear rate (1/s) | Ex. 2 | Ex. 3 | Ex. 4 | Ex. 5 | Ex. 6 |
|---|---|---|---|---|---|
| 1.0000 | 28.99 | 113.60 | 429.50 | 321.00 | 161.00 |
| 10.0000 | 3.75 | 8.51 | 27.29 | 24.43 | 14.80 |
| 100.0000 | 0.83 | 1.36 | 2.60 | 2.11 | 1.79 |

### Example 7

Oil gel example Ex. 3 was used to test the concept of prevent red wine leaking into (under) a clear tray aligner (CTA). A first CTA was filled with the oil gel example Ex. 3 and was then placed over one arch of a corresponding typodont model. A second CTA was not filled with oil gel example and was then placed over the other arch of the same typodont model. The typodont model with CTAs was soaked in red wine for one minute at room temperature. The surface of the CTAs was wiped off and the typodont was assessed. It was visually apparent that red wine had leaked into (under) the CTA which did not have oil gel example applied interiorly. However, there was no wine visible under the CTA which had been first treated with oil gel example Ex. 3 inside. The oil gel example had prevented the ingress of red wine between the typodont teeth and the interior surface of the CTA. The experiment was repeated with the soaking time extended to 24 hours at room temperature. The results were the same; red wine had leaked into (under) the CTA which did not have oil gel example applied interiorly; there was no red wine visible under the CTA which had been first treated with oil gel example Ex. 3 inside.

### Example 8

The experiment of Example 7 was repeated using oil gel example Ex. 4, which contained sodium fluoride. The results of Example 8 were the same as Example 7, the oil gel prevented the ingress of red wine into (under) the clear tray aligner (CTA) even after 24 hours of soaking in red wine there was no red wine visible between the typodont teeth and the interior surface of the CTA.

The above examples demonstrated the following. The rheological property of the oil gel is significant for material handling and application. The gel did not flow at very low shear rate and yet where it is applied in a clear tray aligner (CTA), it did flow easily when there was a high shear such as during pushing the CTA onto the typodont teeth. Excess amount of gel was easily squeezed out of CTA. The oil gel example compositions filled into the CTA and prevented other liquids leaking into the gap between CTA and teeth. Furthermore, the oil provides a coating on teeth, which separate the teeth with the oral environment. Oil is a natural preservative, there is no microbial growth in oil. Therefore, the oil gel can provide the ability to inhibit the growth of microbial on the CTA and on teeth. This can provide a further benefit to the teeth.

1. A system, comprising:
a dental appliance comprising a polymeric shell with a plurality of cavities for receiving one or more teeth; and
a non-aqueous composition disposed in one or more of the cavities of the polymeric shell of the dental appliance, the non-aqueous composition comprising:
an oil; and
a viscosity modifier;
wherein the non-aqueous composition has a viscosity of between 20 and 600 Pa*s at a shear rate 1/s.

2. The system of claim 1, wherein the non-aqueous composition includes an organic fluid in an amount of at least 70 wt. %, based on the total weight of the non-aqueous composition.

3. The system of claim 1, wherein the oil comprises an edible oil.

4. The system of claim 1, claims, wherein the edible oil is cosmetic.

5. The system of claim 1, wherein the oil comprises a plant oil.

6. The system of claim 1, wherein the oil is present in the non-aqueous composition in an amount of between 65 and 95 wt. %, based on the total weight of the non-aqueous composition.

7. The system of claim 1, wherein the viscosity modifier comprises an oil compatible polymer, silica compound, cellulose derivative, or organic modified clay.

8. The system of claim 1, wherein oil compatible polymers are present in the non-aqueous composition in an amount of between 1 and 15 wt. %, based on the total weight of the composition.

9. The system of claim 1, wherein silica compounds are present in the non-aqueous composition in an amount of between 1 and 15 wt. %, based on the total weight of the composition.

10. The system of claim 9, wherein the silica compounds comprise fumed silica.

11. The system of claim 1, wherein the viscosity modifier is present in the non-aqueous composition in an amount sufficient for the non-aqueous composition to form a gel.

12. The system of claim 1, wherein the non-aqueous composition exhibits shear thinning behavior.

13. The system of claim 1, wherein the non-aqueous composition has a refractive index of between 1.20 and 1.65, and the refractive index of the polymeric shell differs from that of the nonaqueous composition by less than 30%.

14. The system of claim 1, wherein the polymeric shell is a polymeric material selected from polyurethane, polyester, polyethylene terephthalate, polyethylene terephthalate glycol, poly cyclohexylenedimethylene terephthalate glycol, poly(meth)acrylates and mixtures and combinations thereof.

15. The system of claim 1, wherein the non-aqueous composition is at least substantially colorless and at least substantially transparent to visible light with a wavelength of 400-750 nm.

16. The system of claim 1, wherein the polymeric shell and the non-aqueous composition transmit at least 60% of incident light with a wavelength of about 400-750 nm.

17. The system of claim 1, wherein the non-aqueous composition has a pH of about 6.0 to about 8.0.

18. The system of claim 1, wherein the non-aqueous composition further comprises a tooth re-mineralizing agent.

19. The system of claim 1, wherein at least one cavity in the polymeric shell of the dental appliance is configured to adjust a tooth from a maloccluded position to a desired position.

20. A method for using the system of claim 1, to adjust a tooth of a patient from a maloccluded position to a desired position, the method comprising placing the dental appliance over the teeth of the patient.
